(19) United States
(12) Patent Application Publication
(10) Pub. No.: US 2022/0244240 A1
(43) Pub. Date: Aug. 4, 2022

(54) CARTRIDGE SYSTEM FOR ANALYTE MEASUREMENT IN A POINT OF CARE SETTING

(71) Applicant: SannTek Labs Inc., Kitchener (CA)

(72) Inventor: Thomas Dunlop, Kitchener (CA)

(21) Appl. No.: 17/166,905

(22) Filed: Feb. 3, 2021

(51) Int. Cl. G01N 33/49; A61B 5/1486; G01N 33/487; G01N 27/327

(52) U.S. Cl. CPC G01N 33/4925; G01N 27/3271; G01N 33/48778; A61B 5/1486

(57) ABSTRACT

A cartridge format for point-of-care analyte detection in a sample of bodily fluid is provided. The cartridge includes a microfluidic system of channels of varying length containing lyophilized reagent for sample processing. Sample loaded into the cartridge moves to the sensor in a generally downward direction via the microfluidic system. The sample reconstitutes the lyophilized reagents within the channels, and is the only fluid required for analyte measurement. Movement of the sample is regulated by two forces—gravity and pressure within the microfluidic system. The cartridge includes one or more vent valves for relieving the pressure in the microfluidic system, thus allowing, or restricting flow of sample and/or reagent through the cartridge by force of gravity. The cartridge may be connected to an analyzer device having one or more actuators for opening and closing the vent valves and a sensor for measuring the analyte.

### TECHNICAL FIELD

The embodiments disclosed herein relate to biosensing and analyte detection, and, in particular to a cartridge system and apparatus for detecting and quantifying a target analyte from a sample in a point of care setting.

### INTRODUCTION

Approaches directed to the detection of analytes such as hormones, disease biomarkers and other chemical species in individuals are important to the promotion of safety and health among individuals and populations. Effective testing for the presence of analytes linked to disease or other physiological conditions may be critical to ensure the health and safety of individuals. Early and effective detection can be critical to successful treatment and health management of populations.

In many circumstances, existing analyte detection techniques utilize laboratory-based testing. Such techniques are often applied by medical professional and require the patient to attend a clinic, hospital, or other healthcare setting. This can be inconvenient to the individual, time-consuming to perform the test and return results, and use invasive practices such as drawing blood.

Further, in some cases, point of care tests (e.g. tests taken at home by users) may be inadequate. Such tests, such as for example testing for luteinizing hormone (LH) to predict ovulation, may not account for considerable variation in hormone levels between individuals. For example, due to extremely high or low baseline LH levels, 1/10 women cannot use today's tests.

Systems utilizing disposable cartridges for testing point of care samples have emerged to address the need to simplify and contain the laboratory components needed for testing into a small form factor that can be used in point of care settings. Such systems typically include a detector (or measurement device) into which the cartridge containing the sample is inserted to obtain a measurement. A limitation of such systems is the need for some means, for example, a pump, motor, piston, diaphragm, air line, etc. to move the sample and reagents through the cartridge. The existence of such components add complexity and expense to the construction and maintenance of existing point of care cartridge testing systems.

It is therefore desired to provide improved systems and apparatus for point-of-care biosensing and analyte detection. In particular, analyte detection systems and devices are desired that reduce inconvenience and expense, such as by enabling use by non-medical professionals at home or other point of care setting and the utilization of small patient samples. Further, in some cases, it is desired that such systems and methods be able to detect an analyte at low levels in the patient sample.

Accordingly, there is a need for systems, methods, and devices for point of care biosensing that overcome at least some of the disadvantages of existing biosensing techniques.

### SUMMARY

According to some embodiments, there is a cartridge system and apparatus for point-of-care measurement of an analyte in a sample of bodily fluid. The system comprises a sample cartridge and an analyzer device. The cartridge may be employed as a one-use cartridge.

The cartridge includes an inlet for receiving the sample and a reservoir in fluidic connection with the inlet. A volume of sample is added to the cartridge via the inlet and drains into the reservoir by force of gravity.

The cartridge may include an electrochemical sensor for detecting the analyte. The electrochemical sensor contacts the sample within the reservoir. The cartridge includes electrical contacts disposed on an external surface for interfacing with the analyzer device.

The cartridge includes a first channel having a first length, wherein the first channel is in fluidic connection with the inlet and the reservoir. The cartridge includes a second channel have a second length, wherein the second channel is in fluidic connection with the inlet and the reservoir. The cartridge includes a first vent valve for regulating the pressure upstream of the reservoir, wherein opening the vent valve relieves pressure in the first channel and the second channel thereby draining fluid from the inlet into the reservoir via the first channel and the second channel, by force of gravity.

The first and second channels may contain lyophilized reagents that are reconstituted by the sample flowing through the cartridge.

The analyzer device includes a first stopper for opening and closing the first valve vent. The stopper comprises a body and a needle protruding from the body. The analyzer device includes a first actuator for moving the first stopper between a first position to close the first vent valve and a second position to open the first vent valve. The first stopper aligns with the first vent valve when the cartridge is connected to the analyzer device.

The cartridge includes a waste channel in fluidic connection with the reservoir, and a second vent valve for regulating the pressure in the waste channel. Opening the second vent valve relieves the pressure in the waste channel thereby draining fluid from the reservoir into the waste channel by force of gravity. The analyzer device may include a second stopper for opening and closing the second vent valve and a second actuator for moving the second stopper.

The analyzer device includes a potentiostat for processing the signals from the electrochemical sensor to calculate a measurement of the analyte. The analyzer device includes control electronics configured to drive the first actuator and the second actuator in a predefined sequence to process the sample and perform a measurement of the analyte.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a block diagram of a system for analyte detection, according to an embodiment;

FIG. 2A is a perspective view of a sample cartridge, according to an embodiment;

FIG. 2B is an exploded view of the cartridge in FIG. 2A;

FIG. 2C is a top view of the base and microfluidic system of the cartridge in FIGS. 2A-2B;

FIGS. 3A-3B are top and front views, respectively, of a stopper, according to an embodiment;

FIG. 3C is a perspective view the stopper in FIGS. 3A-3B, shown in relation to the sample cartridge in FIG. 2A;

FIG. 3D is a diagram of region A in FIG. 3C showing the stopper in a starting position relative to a vent valve;

FIG. 3E is a diagram of region A in FIG. 3D showing stopper and the vent valve in an open position;

FIG. 3F is a diagram of region A in FIG. 3D showing the stopper and the vent valve in a closed position;

FIG. 4A is a perspective view of a sample cartridge, according to an embodiment;

FIG. 4B is an exploded view of the sample cartridge in FIG. 4A;

FIG. 4C is a top view of the base and microfluidic system of the sample cartridge in FIGS. 4A-4B;

FIG. 4D is a bottom view of the base and microfluidic system of the sample cartridge in FIG. 4A-4B;

FIG. 5A-5B are perspective and top views, respectively, of a sample cartridge, according to an embodiment; and

FIG. 6 is a top view of a base and microfluidic system for a sample cartridge, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

The following relates generally to systems and apparatus for biosensing, and more particularly to a system, method, and apparatus for detecting and quantifying concentrations of an analyte from a liquid sample in a point of care setting. The liquid sample may be a bodily fluid. The system provides a detection platform for a myriad of analyte detection and measurement applications, for example, detecting the level of LH in a urine sample to predict ovulation.

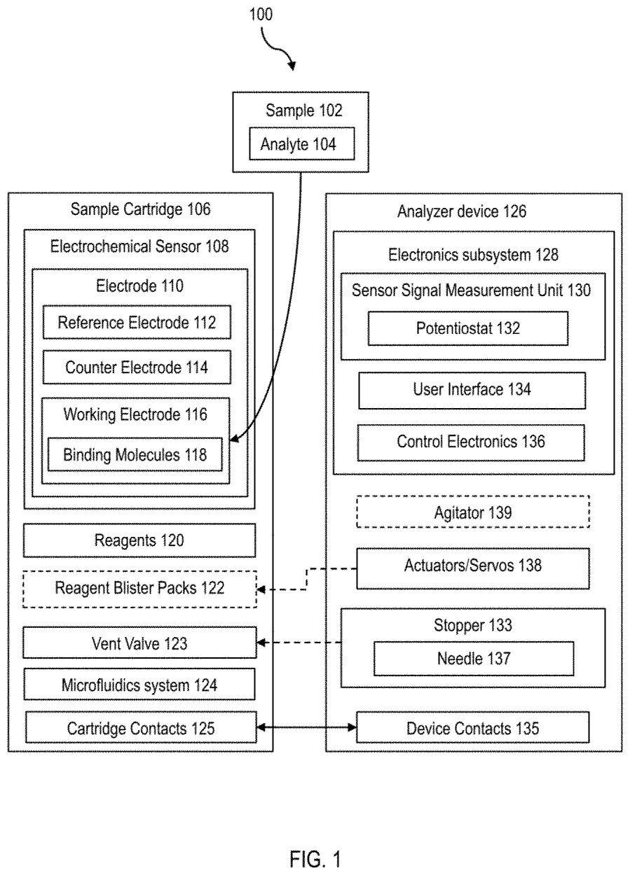

Referring to FIG. 1, illustrated therein is a system 100 for detecting an analyte 104 in a sample 102, according to an embodiment. The sample 102 is a bodily fluid, for example, urine, saliva or blood. The system 100 is used to process and analyze the sample 102 to determine the presence of the analyte 104.

The analyte 104 is a biological molecule detected by the system 100 and which may be present in the sample 102 depending on the condition of the sample provider. The analyte 104 may be a hormone, such as testosterone or luteinizing hormone (LH). The analyte 104 may be an infectious disease marker, such as a viral antigen or an antibody.

The system 100 includes a sample cartridge 106 and an analyzer device 126. Generally, the sample cartridge 106 can be connected to (e.g. inserted into) or otherwise interfaced with the analyzer device 126. The analyzer device 126 may include a slot or opening for inserting the sample cartridge 106 into the analyzer device 126.

The sample cartridge 106 may be a one-use disposable cartridge. The sample cartridge 106 houses the electrochemical sensor 108, reagents 120 and a microfluidics system 124 that may be adapted to automate steps that, using existing methods, would be performed in the laboratory. This may advantageously allow the system 100 to be easily used by individuals having little or no specialized training in a point of care setting.

The system 100 may use an electrochemical biosensor 108 to detect the analyte 104. According to other embodiments, the system 100 may use a fluorescence or colorimetric sensor for detecting the analyte 104.

Generally, the electrochemical sensor 108 and the electrode 116 may be of a type or format disclosed in US provisional patent application 63/057,230. For example, the electrochemical sensor 108 may be an electrochemical immunosensor with an in-solution redox probe; the electrochemical sensor 108 may be an electrochemical immunosensor with a self-assembled monolayer and immobilized redox probe or enzyme; the electrochemical sensor 108 may be a faradic or a non-faradaic electrochemical immunosensor; the electrochemical sensor 108 may be an electrochemical immunosensor implementing a sandwich electrochemical immunoassay; or the electrochemical sensor 108 may be an electrochemical immunosensor implementing a competitive electrochemical immunoassay.

The electrochemical sensor 108 includes an electrode 110. The electrode 110 may be a conductor through which electricity enters or leaves the sensor 108. The electrode 110 includes three sub-electrodes including a reference electrode 112, a counter electrode 114, and a working electrode 116. In an embodiment, the working and counter electrodes 114, 116 are composed of gold and the reference electrode 112 is composed of silver. During operation of the system 100, the electrode 110 is in contact with a testing solution which includes the sample 102 (which may be processed).

The working electrode 116 applies a desired potential or current in a controlled manner. The working electrode 116 may facilitate a transfer of charge to and from the testing solution (e.g. in an impedimetric format). The reference electrode 112 acts as a reference in measuring and controlling the potential of the working electrode 116. The counter electrode 114 passes current to balance the current observed at the working electrode 116.

The electrochemical sensor 108 further includes a plurality of binding molecules 118 attached to a surface of the working electrode 116 which is exposed to the sample 102. The binding molecule 118 may be a receptor molecule specific for the analyte 104 or may be the analyte 104 itself acting in a competitive capacity (i.e. a competitive analyte in a competitive assay format). For example, in a Luteinizing Hormone (LH) detection application, the binding molecule 118 may be LH or a LH-specific receptor.

The binding molecules 118 may be attached to the working electrode 116 using a self-assembled monolayer (SAM). According to some embodiments, the working electrode 116 may also include a redox probe attached thereto via a SAM. According to other embodiments, wherein the sensor is fluorescence or colorimetry-based (i.e. having no electrode 110), the binding molecules 118 may be located within the microfluidics system 124.

The sample cartridge 106 also contains reagents 120. The reagents 120 may be one or more liquid phase or dried (e.g. lyophilized) components which are used by the detection system 100. The reagents 120 may include a redox reagent including a redox probe. The reagents 120 may include any one or more of an anticoagulant, a buffer, or other pH modulating reagent.

The reagents 120 may include a label for binding to the analyte 104 and detecting the analyte 104 in a sandwich or competitive immunoassay. For example, in a sandwich immunoassay the label may be: an enzyme-labelled secondary antibody and reactive reagent (e.g. tetramethylbenzidine (TMB) and H₂O₂) to be catalyzed by the enzyme (e.g. horseradish peroxidase, HRP); a fluorescently-labelled secondary antibody; or a colorimetric indicator-labelled secondary antibody and detection solution.

The sample cartridge 106 may include reagent blister packs 122 for storing liquid phase reagents 120. According to some embodiments, wherein the reagents 120 are lyophilized, the sample cartridge 106 may not include reagent blister packs 122.

The sample cartridge 106 includes a microfluidic subsystem 124. The microfluidic subsystem 124 transports and holds the sample 102 at multiple stages of the analysis. The fluidics subsystem 124 includes a network of fluidic channels and reservoirs. The channels facilitate movement of the sample 102 (and/or reagents as the case may be) through the sample cartridge 106 during sample processing and analyte measurement. The reservoirs contain elements (e.g. the electrochemical sensor 108) that reagents 120 interact with during sample processing.

The movement of the sample 102 and reagents through the cartridge 106 is regulated by 2 forces: gravity and pressure. Generally, gravity flows the sample 102 and reagents downward through the cartridge 106, and pressure in the microfluidic subsystem 124 restricts the flow of the sample 102 and reagents through the cartridge 106.

The sample cartridge 106 includes one or more vent valves 123 in fluidic connection with the microfluidic subsystem 124. The opening and closing of the vent valves 123 to allow or prevent, respectively, the release of air from the microfluidic subsystem 124 changes the pressure within the microfluidic subsystem 124. The change in pressure causes the sample 102 (and/or reagents 120 as the case may be) to move through the sample cartridge 106 by force of gravity without requiring a pump, piston, or the like to move the sample 102, as described below.

The sample cartridge 106, includes cartridge contacts 125 for contacting complementary device contacts 126 on the analyzer device 126. For example, the analyzer device 126 may connect to the sensor cartridge 106 and facilitate generating and analyzing a detection signal by interfacing the respective contacts 125, 135 of the sensor cartridge 106 and analyzer 126. The cartridge contacts 125 are disposed on an external surface of the sample cartridge 106 such that when the sample cartridge 106 is connected to (i.e. inserted into) the analyzer device 126, the cartridge contacts 125 come into contact with the device contacts 135. Upon establishing the interface, the cartridge contacts 125 may relay signals between the electrochemical sensor 108 and the electronics subsystem 128 in the analyzer device 126.

The electronics subsystem 128 includes a sensor signal measurement unit 130, a user interface 134 and control electronics 136. The control electronics 136 controls one or more actuators/servos 138, an agitator 139 and sensors (not shown).

The agitator 139 may be DC brush motor for mobile devices, or the like, that vibrates when a voltage is applied. Agitation may be performed at various stages of sample preparation and analyte measurement when the cartridge 106 is connected to the analyzer device 126. The vibrations from the agitator 139 are transferred to the sample cartridge 106 by contact when the cartridge 106 is connected (i.e. inserted) to the analyzer device 126.

The actuators/servos 138 may include one or more of a servo motor or a linear actuator. The control electronics 136 includes driver circuitry to power the actuators/servos 138 and agitator 139 in the required timing to process the sample 102 and measure the analyte 104 (e.g. a sequence of pulses to drive a servo motor or linear actuator, opening/closing vents valves 123 in sequence, etc.).

According to some embodiments, the actuators/servos 138 include a linear stepper motor configured to compress the reagent blister pack 122 to disgorge the reagent 120 contained therein. The actuator/servos 138 may be positioned to align, or make contact with, a reagent blister pack 112 on the cartridge 106 when the cartridge 106 is connected to (i.e. inserted into) the analyzer device 126.

According to some embodiments, the actuators/servos 138 are configured to move a stopper 133 and needle 137 to open/close vents valves 123 in the sample cartridge 106 to control sample 102 flow through the cartridge 106. The stopper 133 may be positioned to align with a vent valve 123 when the cartridge 106 is connected (i.e. inserted) to the analyzer device 126.

According to some embodiments, the analyzer device 126 may include more than one stopper 133. Generally, the analyzer device 126 will include a stopper 133 for each vent valve 123 in the sample cartridge 106.

According to some embodiments, the analyzer device 126 may include one or more of an optical sensor, a switch, or an actuator feedback sensor (e.g. measuring the load experienced by a motor by measuring the current it consumes). The sensors may utilize dedicated electronics to pre-process or amplify their signals. Signals from sensors are digitized by an analog to digital (ADC) converter. This may allow the signals to be used by firmware or software of the analyzer device 126.

To execute a test (i.e. measure the analyte 104 in the sample 102), an algorithm implemented in firmware of the analyzer device 126 may activate the actuators 138 in a particular sequence. The analyzer device 126 may use readings from the sensors to modify the actuators/servos 138 sequence as the test progresses. The algorithm may also use measurements from the potentiostat 132 as a sensor input to modify the actuators/servos 138 sequence.

The sensor signal measurement unit 130 includes a potentiostat 132. The potentiostat may apply variable potentials to the working electrode 116 relative to the reference electrode 112 while measuring the current that flows as a result of the electrode 110 reaction. The potentiostat 132 may be an off-the-shelf potentiostat chip, such as an AD5941 chip from Analogue Devices. The potentiostat 132 may be a Biologic SP-150.

The sensor signal measurement unit 130 also includes a processor (not shown) for executing an analyte measurement module configured to determine an analyte level for the sample 102 based on a detection signal generated by the system 100.

Generally, the signal measurement unit 130 is configured to apply a voltage scan using the potentiostat 132. In an embodiment, this may include generating a differential pulse voltammetry (DPV) output signal data and generating an analyte level using the DPV output signal data. The signal measurement unit 130 may determine the presence of a redox peak in the DPV output signal data. The analyte 104 level may then be determined by the signal measurement unit 130 using the determined peak. The system 100 may be configured to operate quantitatively such that if enough analyte 104 is present to block all binding sites, no signal is generated, if no analyte 104 is present, then a full signal is generated, and if enough analyte 104 is present to only block half the binding sites, a signal with half the intensity of the full signal is generated. In this way, various concentrations of analyte 104 can be distinguished from one another by the signal measurement unit 130.

According to another embodiment, the signal measurement unit 130 is configured to employ chronoamperometry (CA) using the potentiostat 132 to determine an analyte level in the sample 102 (e.g. for an enzyme-based sensor implemented by the system 100 using an enzyme label such as horseradish peroxidase). CA may be used in a sandwich sensor format or a competitive sensor format. The signal measurement unit 130 may be configured to store a plurality of CA plot data for various known concentrations of the enzyme label. The CA plot data is the current (generated by the enzyme label reacting with redox probe at the electrode), as a function of time, at a constant voltage. The signal measurement unit 130 may be configured to determine and store such plot data values and perform a concentration or other determination using the stored plot data values to provide an indication of analyte level.

According to other embodiments, wherein the analyte 104 is detected by fluorescence or colorimetry rather than an electrochemical sensor 108, the signal measurement unit 130 may include a photodiode or a camera (not shown) for detecting fluorescence or color change, respectively. In embodiments wherein the analyte is detected by fluorescence, the analyzer device 126 further includes a light source (i.e. an electromagnetic radiation emitter) for illuminating the sample.

The analyzer device 126 includes a user interface software module 134. The user interface software generates a user interface screen using input from the electronics subsystem (i.e. the analysis results). The user interface screen may include one or more user interface elements configured to receive input data from a user of the system 100.

The analyzer 126 includes a display (not shown). The display may be an LCD screen. The display is connected to the electronics subsystem 128. The display is configured to render a display of one or more user interface screens generated by the user interface software 134. In an embodiment, the user interface may be displayed on a user terminal such as a cell phone. The user terminal may connect to the electronics subsystem 128 by wireless connection, such as Wi-Fi or Bluetooth.

The analyzer device 126 does not include a pump, or other means, for transporting the sample 102 through the sample cartridge 106 during sample 102 processing and analyte 104 measurement. Accordingly, the overall size and complexity of the analyzer device 126 can be reduced to a form factor that is easily portable and practical for point of care testing (i.e. at-home testing). The analyzer device 126 may have a wired or wireless power supply or be powered by an internal battery (not shown).

The system 100 may have various analyte detection applications, some of which will now be described by way of example.

In an embodiment, the system 100 may implemented for a luteinizing hormone (LH) test using a urine sample. The system 100 may help women looking to get pregnant track their ovulation cycle. LH peaks in urine and blood one day before peak fertility. Existing approaches measure whether or not LH is above a certain threshold to identify the LH spike. Baseline and peak LH levels can be very variable among women. For example, one in ten women has a LH peak lower than the average LH baseline and cannot use threshold-based products. However, such women still have a LH peak greater than their baseline. The system 100 performs quantitative testing for LH. By providing a quantitative test for LH, the system 100 may work for a wider variety and range of women no matter the magnitude of their baseline or peak.

In an embodiment, the system 100 may be implemented or at-home (i.e. point of care) fertility testing. The system 100 may test for various hormones in a urine sample to assist couples in generating a pregnancy. Such an embodiment of the system 100 may advantageously replace existing methods such as vaginal thermometers and blood tests used today. The system 100 and the test implemented thereby may be used as a precursor or replacement for visiting a fertility clinic. In such an embodiment, the analyte 104 may be estrogen, luteinizing hormone, follicle stimulating hormone, or progesterone.

In an embodiment, the system 100 may implement hormone testing for endocrinologists and fertility clinics. The system 100 is configured to test for various hormones in a urine sample for use in endocrinologist offices and fertility clinics. This may allow for faster reporting of results and potentially higher throughput. The testing may advantageously be less invasive for patients than a traditional blood draw. The analyte 104 detected by the system 100 may be any one or more of androstenedione, DHEAS, estradiol, free beta HCG, FSH, hCG, LH, PAPP-A, progesterone, prolactin, SHBG, testosterone, and unconjugated estriol.

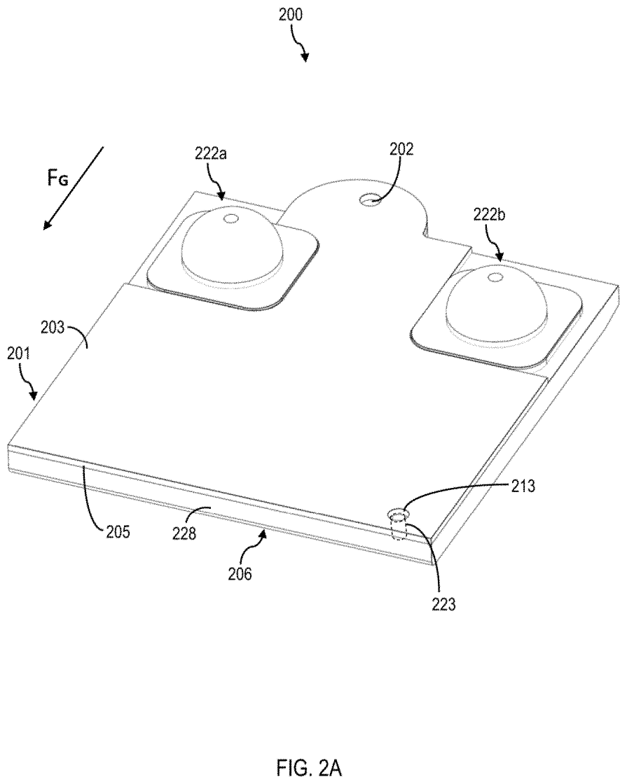

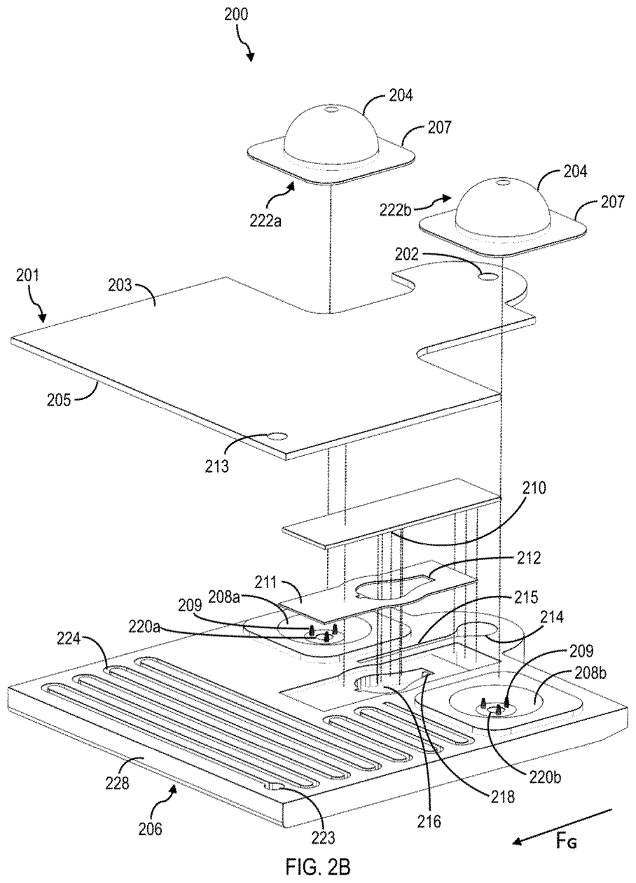

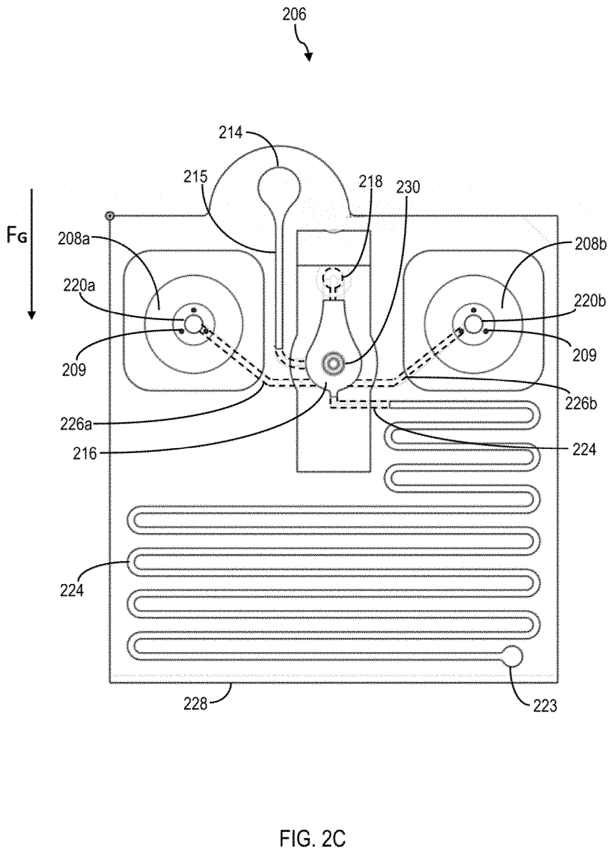

Referring to FIG. 2A, illustrated therein is a sample cartridge 200 according to an embodiment. The cartridge 200 may be the sample cartridge 106 in FIG.

The cartridge 200 includes an inlet 202 for receiving a sample of bodily fluid, for example, urine. Sample may be injected into cartridge 200 via the inlet 202 using a pipette, or the like. The inlet 202 may be a capillary action port for drawing sample into the cartridge 200. According to some embodiments, the inlet 202 may include a sample processing module (not shown). The sample processing module may be specific to the sample, analyte or application the cartridge 200 is used for. For example, in embodiments wherein the sample is blood, the sample processing module may transform whole blood added to the inlet 202 into plasma or serum using a filter membrane or electroosmotic flow.

According to some embodiments, the inlet 202 may include a protuberance (not shown) which may be dipped into a volume of sample (e.g. urine in a cup). The sample flows into the protuberance and fills up a channel within the protuberance by capillary force. The cartridge 200 is then inverted, causing the sample in the protuberance to be pulled through the inlet 202 and into the cartridge 200 by force of gravity. For reference, arrow FG points in the direction of gravitational force when the cartridge is inverted. The protuberance may be constructed of a clear material such as clear plastic or glass to enable the user to observe the volume of sample taken into the protuberance.

The cartridge includes a base 206. The base 206 contains a system of microfluidic channels and reservoirs along which the reagents and sample move through the cartridge 200 during sample processing and analyte measurement.

The cartridge 200 includes a hydrophilic cover 201 having a top surface 203 and a bottom surface 205. The cover 201 includes a recess 213 in the top surface 203 that extends to the bottom surface 205. According to other embodiments, in place of the recess 213, the cover 201 includes an opening between the top and the bottom surfaces 203, 205. The cover 201 may be optically transparent.

The cartridge 200 includes one or more reagent blister packs 222 for storing reagents. Each blister pack 222 stores liquid phase reagents for sample processing. The type and amount of reagent in each blister pack 222 may be specific to the analyte measured or application the cartridge 200 is used for. For example, the reagent blister pack 222 may contain a redox probe, a wash buffer, etc.

Referring now to FIG. 2B, shown therein is an exploded view of the sample cartridge 200.

The bottom surface 205 of the cover 201 includes adhesive to attach the cover 201 to the base 206. When the cover 201 is attached to the base 206, the bottom surface 205 encloses the channels and reservoirs within base 206. The bottom surface 205 is hydrophilic to promote the flow of reagents and sample through the cartridge 200 along the channels and reservoirs formed between the base 206 and cover 201. According to some embodiments, the cover 201 may be constructed of PET and heat bonded directly to the base 206 without adhesive.

Each blister pack 222 is constructed of metalized PET and includes a compressible dome 204 and a permeable base 207 enclosing a volume of reagent. The blister pack 222 is attached adjacent to a well 208 in the base 206. The well 208 includes one or more spikes 209, such that the permeable base 207 contacts the spikes 209 within the well 208 when the blister pack 222 is attached to the base 206.

To release the reagent from the blister pack 222, the dome 204 is compressed by mechanical means. For example, when the cartridge 200 is inserted into an analyzer device (i.e. analyzer device 126 in FIG. 1) an actuator within the analyzer device may compress the dome 204. Compressing the dome 204 causes the spikes 209 to penetrate the permeable base 207 and disgorge the reagent into the well 208. The more the dome 204 is compressed, more volume of reagent is disgorged into the well 208.

The cartridge 200 includes an electrode 210 having a ceramic or PET substrate. The electrode 210 may be the electrode 110 in FIG. 1. The electrode 210 may be an off-the-shelf electrode, for example, DropSens DRP-220AT. The electrode 210 is affixed to the base 206 by a double-sided hydrophobic rubberized adhesive 211. The adhesive 211 includes a cutout 212 in the region of the electrode 210. According to other embodiments, the electrode 210 may be heat bonded directly to the base 206 without adhesive 211.

The base 206 of the cartridge 200 includes a microfluidic system including a network of reservoirs, channels and vents. The microfluidic system is configured for a sample to pass through the cartridge 200 by force of gravity without requiring a pump, air line, or other means to transport the sample when the cartridge 200 is inserted into an analyzer device. The passage of the sample through cartridge 200 is controlled by the release of air (and change in pressure) in the microfluidic system by opening and closing of a vent valve 223 as described below.

The base 206 includes an inlet reservoir 214 in fluidic connection with the inlet 202. Sample enters the cartridge 200 via the inlet 202 and collects in the inlet reservoir 214.

The base 206 includes a sensor reservoir 216. The sensor reservoir 216 is in fluidic connection with the inlet reservoir 214 via an inlet channel 215. The electrode 210 is exposed to the sample within the sensor reservoir 216. For example, the electrode 210 may form a surface (or a cover at least a portion of the surface area) of the sensor reservoir 216, as shown, whereby sample within the sensor reservoir 216 may come into contact with the electrode 210 through the cutout 212 in the adhesive 211. According to some embodiments, the electrode 210 may be positioned entirely within the sensor reservoir 216.

The sensor reservoir 216 contains the binding molecules (i.e. binding molecules 118 in FIG. 1) that are specific to the analyte. The binding molecules may be immobilized on the electrode 210 as described above. The sensor reservoir 216 may also contain dried label for forming a sandwich complex with the analyte and the binding molecule. The label may be deposited on the electrode 210 substrate, directly on the working electrode surface, or on a divot 230 adjacent to the electrode 210. According to other embodiments, wherein the sensor is fluorescence or colorimetry-based (i.e. having no electrode 210), the binding molecules may be immobilized on a glass plate in place of the electrode 210, and the label may be deposited on a surface of the reservoir 216.

The base 206 includes a waste channel 224 for sample (and/or reagent as the case may be) to drain out of the sensor reservoir 216. The waste channel is 224 is fluidly connected with the sensor reservoir 216 and a vent valve 223. The vent valve 223 releases the pressure in the waste channel 224 by allowing air through the vent valve 223. When the vent valve 223 is closed, backpressure in the waste channel 224 counteracts gravity to prevent the sample/reagent from draining out of the sensor reservoir 216 or, generally, prevents downward flow of the sample/reagent through the cartridge 500.

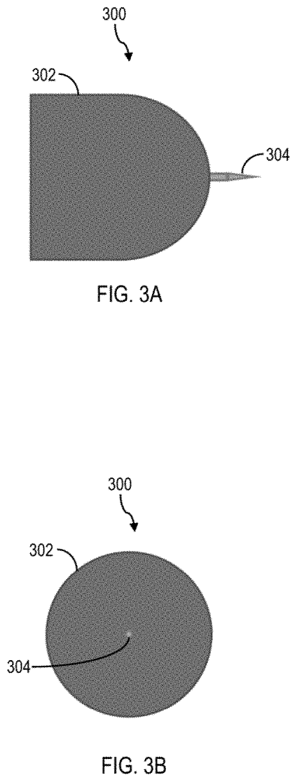

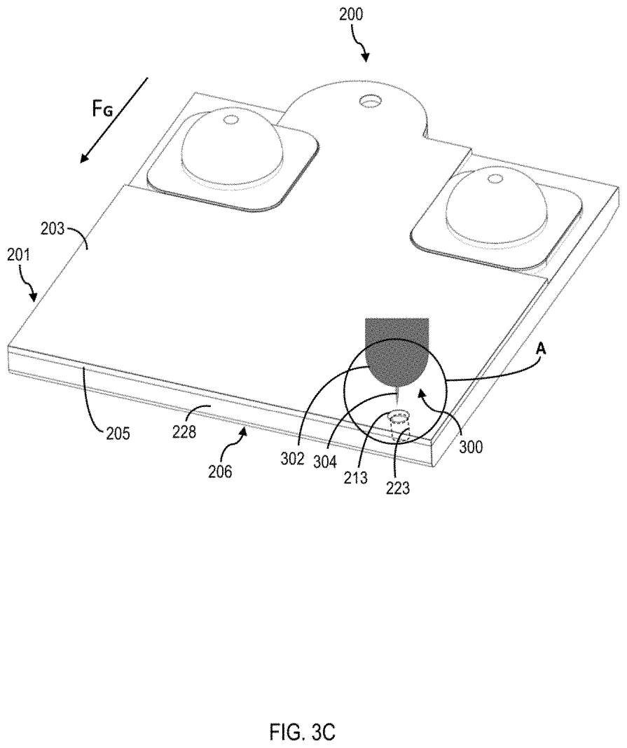

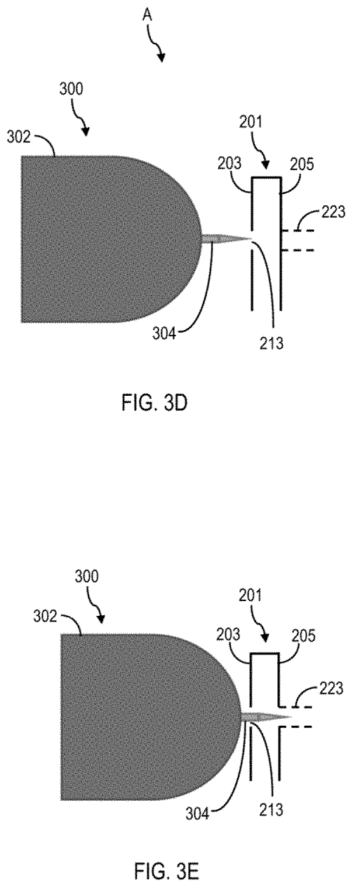

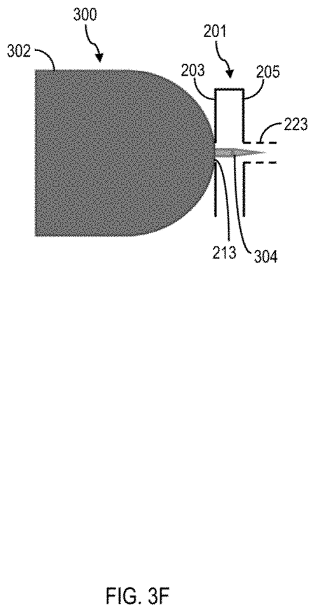

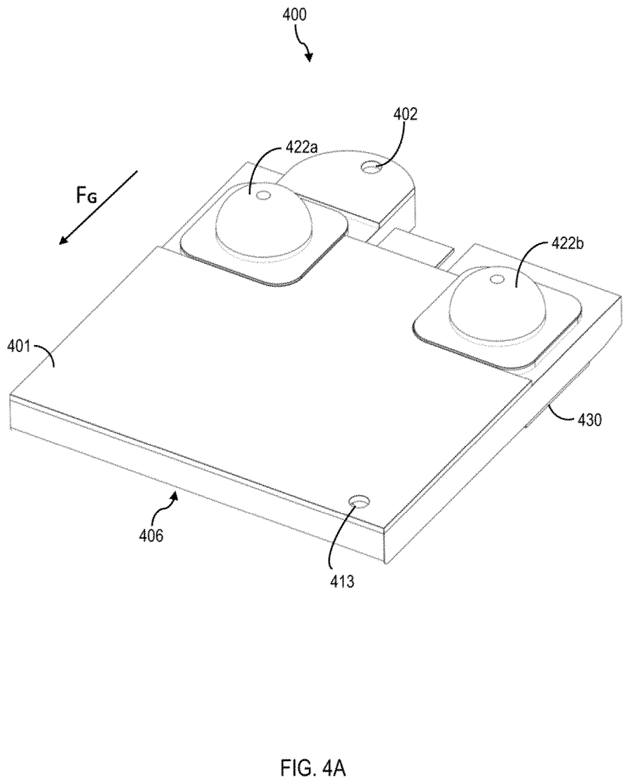

The vent valve 223 is aligned with the recess 213 in the cover 201 such that the bottom surface 205 of the cover 201 seals the vent valve 223 when the cover 201 is attached to the base 206. The bottom surface 205 of the cover 201 within the recess 213 may be pierced (as shown in FIGS. 3A-3F) to open the vent valve 223.

Referring now to FIG. 2C, shown therein a top view of the base 206. The base 206 is constructed of a single piece of PET or similar material formed by injection molding or 3D printing. The base 206 may be optically transparent. The base 206 contains the microfluidic system including the reservoirs 214, 216, channels 215, 224, 226a, 226b, openings 220a, 220b and vents 218, 223. The sections of the channels 215, 224, 226a, 226b embedded within the base 206 are indicated with dashed lines.

Referring to FIGS. 2A-2C, an exemplary implementation of the cartridge 200 for detecting presence of Luteinizing Hormone (LH) in a sample of urine is described.

Urine is taken into the cartridge 200 via the inlet 202. The cartridge 200 is then inverted and connected to (i.e. inserted into) an analyzer device (i.e. device 126 in FIG. 1). The cartridge 200, when inserted into the analyzer device, must be oriented with the inlet 202 upward and a bottom end 228 downward so that the sample travels through the cartridge 200 in a generally downward direction from the inlet 202, to the sensor reservoir 216, to the vent valve 223 by force of gravity (indicated by arrow FG).

Gravity drains the urine from the inlet 202 into the inlet reservoir 214, and then into the sensor reservoir 216 via the inlet channel 215. When urine reaches the sensor reservoir 216, there are four possible paths to take.

The first two paths are into the reagent channels 226a, 226b. These paths are blocked when the reagent blister packs 222a, 222b are uncompressed and intact. The intact reagent blister packs 222a, 222b seal the openings 220a, 220b to the respective reagent channels 226a, 226b and the backpressure in the sealed reagent channels 226a, 226b prevents flow of urine into the reagent channels 226a, 226b.

The third path is into the waste channel 224. This path is blocked when the vent valve 223 is sealed by the cover 201 creating enough backpressure in the waste channel 224 to prevent urine from flowing in.

The fourth path is to fill the sensor reservoir 216 toward a top vent 218. The top vent 218 is not sealed and thus provides the only path for the urine to flow with no resistance. An appropriate volume of urine should be added to the inlet 202 to ensure the sensor reservoir 216 does not fill up completely and overflow the top vent 218. According to an embodiment, the sensor reservoir 216 may include an overflow opening.

As the urine flows into and fills the sensor reservoir 216, the urine rehydrates a dried label. According to various embodiments, the label may be deposited within the sensor reservoir 216, or along a path taken by the sample to reach the sensor reservoir 216. According to an embodiment, the label may be pre-mixed with the sample (e.g. manually by a user) prior to placing the sample into the cartridge 200.

The urine is then left in the sensor reservoir 216 for an incubation period (e.g. 30 minutes). During this time two processes take place: (1) the urine reconstitutes the dried label ; and (2) the LH in the urine binds with the binding molecules on the electrode 210, (or on a surface of the sensor reservoir 216 according to other embodiments), and the reconstituted label, forming a sandwich complex. Reconstituted label that is unable to bind to LH (because there is not enough LH present in the urine) is left unbound in solution. The cartridge 200 may be agitated during the incubation period to promote process (1) and/or (2).

Following the incubation period, the bottom surface 205 of the cover 201 within the recess 213 is pierced (see FIGS. 3C-3D). When pieced, the vent valve 223 is opened and the backpressure in the waste channel 224 is relieved and urine and most of the unbound reconstituted label flows from the sensor reservoir 216 into the waste channel 224 by force of gravity. The vent valve 223 is left open for enough time to fully drain the fluid from the sensor reservoir 216 into the waste channel 224. The vent valve 223 is then sealed (see FIG. 3E), creating backpressure in the waste channel 224, and the flow of fluid along the waste channel 224 stops almost immediately.

Next, to wash unbound label from the electrode surface 210 and/or the sensor reservoir 216, the reagent blister pack 222b is compressed to release a wash buffer (e.g. phosphate buffered saline, PBS) contained in the blister 222b. The reagent blister 222b may be compressed by an actuator in the analyzer device (i.e. actuator 138 in FIG. 1). As described above, the actuator compresses the dome 204 of the blister pack 222b until the spikes 209 break the permeable base 207 of the blister pack 222b.

Breaking of the blister pack 222b opens the seal on the opening 220b allowing the wash buffer from the blister pack 222b to drain into well 208b and into the reagent channel 226b via the opening 220b. As the blister pack 222b is compressed, the wash buffer is pushed along reagent channel 226b into the sensor reservoir 216. Backpressure from the vent valve 223 being closed prevents wash buffer from flowing into the waste channel 224. Similarly, backpressure prevents the wash buffer from flowing into the reagent channel 226a because the opening 220a is sealed by the blister pack 222a. Thus, wash buffer can only flow into the sensor reservoir 216 taking the path of least resistance towards the top vent 218 to fill the sensor reservoir 216.

Wash buffer is left on the electrode 210 surface for a period of time (e.g. 10 seconds). The cartridge 200 may be agitated while the wash buffer is on the electrode 210. The vent valve 223 is then opened (see FIG. 3D), to relieve backpressure in the waste channel 224, thus allowing the wash buffer and remaining unbound reconstituted label to drain from the sensor reservoir 216 into the waste channel 224 by force of gravity. The wash step may be repeated, one or more times, by closing the vent valve 223, further compressing the reagent blister 222b to disgorge more wash buffer into the sensor reservoir 216 and then opening the vent valve 223 to allow the wash buffer to drain into the waste channel 224 by force of gravity. Following the wash step(s) only reconstituted label that is bound to LH should remain in the sensor reservoir 216. Following the wash step(s) the vent valve 223 is closed.

Next, the reagent blister pack 222a is compressed to release a reagent contained in the blister pack 222a. The reagent in the blister 222a will vary based on the assay format and label used. Generally, the reagent will react with the reconstituted label to indicate the presence of the analyte. For example, according to embodiments wherein the analyte is detected by electrochemistry (i.e. using an electrode 210), and the label is a secondary antibody labelled with an enzyme (e.g. HRP), the reagent may be a redox probe solution (e.g. tetramethylbenzidine (TMB) and H₂O₂) to be catalyzed by the enzyme. The redox probe solution will react with the HRP in the presence of voltage to generate current at the electrode 210.

According to other embodiments wherein the analyte is measured by fluorescence, and the label is a fluorescently-labelled secondary antibody, the reagent in blister pack 222a may be the same wash buffer as contained in blister pack 222b, or an anti-photobleaching reagent to enhance fluorescence of the label. According to other embodiments wherein the analyte is detected by colorimetry, and the label is a colorimetric indicator, the reagent in blister pack 222a may be a detection solution to elicit a color change in the indicator.

Reagent blister pack 222a is compressed in the same manner as described above for reagent blister pack 222b The reagent blister 222a may be compressed by an actuator in the analyzer device (i.e. actuator 138 in FIG. 1). Breaking of the blister pack 222a opens the seal on the opening 220a allowing the reagent solution from the blister pack 222b to drain into well 208a and into the reagent channel 226a via the opening 220a. As the blister pack 222a is compressed, the solution is pushed along reagent channel 226a into the sensor reservoir 216. Pressure from the vent valve 223 being closed prevents the solution from flowing into the waste channel 224. Thus, the solution can only flow into the sensor reservoir 216.

Following addition of the reagent from blister pack 222a, the measurement of the analyte is performed by the analyzer device (i.e. analyzer device 126 in FIG. 1). The measurement may vary according to the assay format and label used. According to embodiments wherein the cartridge includes an electrode 210 for measuring reaction of an enzymatic label with a redox probe, the measurement of LH by chronoamperometry (CA) may be performed by applying a constant voltage across the electrode 210 using the potentiostat (i.e. potentiostat 132 in FIG. 1) and measuring the current

According to other embodiments wherein LH is detected by fluorescence, an emitter in the analyzer device 126 directs a wavelength of radiation into the sample reservoir 216 to be absorbed by the fluorescent label. The fluorescent label then emits a second wavelength of light (i.e. fluorescence) that is measured by a photodiode in the analyzer device. The intensity of the emitted fluorescence light is proportional to the amount of LH present in the sample and is converted to a concentration. Similarly, according to embodiments wherein LH is detected by colorimetry, a camera in the analyzer device counts a number of pixels changing in color to determine the amount of LH present in the sample.

The measurement of the analyte may be performed over a period of time. The cartridge 200 may be agitated during the measurement. After the measurement is obtained, the vent valve 213 is opened and the fluid drains into the waste channel 224 by force of gravity.

Referring to FIGS. 3A-3B, shown therein are top and front views of a stopper 300, according to an embodiment. The stopper 300 may be the stopper 133 in FIG. 1. The stopper includes a body 302 constructed of silicone, rubber, or a similar material. According to some embodiments (as shown), the stopper 300 includes a needle 304 protruding from the body 302.

FIG. 3C shows the stopper 300 in relation to the sample cartridge 200. The stopper 300 is positioned to align with the recess 213 in the cover 201 and the vent valve 223 in the base 206 when the sample cartridge 200 is inserted into the analyzer device. The needle 304 is perpendicular to the cover 201 and centered with respect to the recess 213 and vent valve 223. It should be noted that the sample cartridge 200 is inserted into the analyzer device with end 228 facing downward, and thus the cartridge 200 will be oriented vertically, rather than horizontally as depicted in FIG. 3C (note the direction of gravitational force indicated by arrow FG).

FIG. 3D is a magnified view of region A in FIG. 3C. For ease of illustration some structures have been omitted. FIG. 3D shows the stopper 300 at a starting position. The starting position is generally the position at which the stopper 300 is in relation to the cartridge 200 when the cartridge is inserted into the analyzer device. In the starting position, the needle 304 is adjacent to the recess 213. The vent valve 223 is sealed by the bottom surface 205 of the cover 201.

The stopper 300 may be moved by an actuator or servo (not shown) to open or close the vent valve 223. The actuator or servo may be actuator/servo 138 in FIG. 1. Generally, moving the stopper 300 to press against the cover 201 will close the vent valve 223; moving the stopper 300 away from the cover 201 will open the vent valve 223.

FIG. 3E shows the valve vent 223 in an open position. To open the valve vent 223, the stopper 300 is moved by an actuator/servo connected to the stopper. The stopper 300 is moved toward the cover 201, such that the needle 304 passes through the recess 213 and pierces the bottom surface 205 of the cover 201. After piercing the cover 201, the stopper 300 withdraws to the starting position. With the cover 201 pierced, air can be expelled through the vent valve 223.

FIG. 3F shows the vent valve 223 in a closed position. To close the vent valve 223, the stopper 300 is moved by the actuator to block the recess 213. The stopper 300 is moved toward the cover 201 until the body 302 presses against the recess 213 to form an airtight seal. The airtight seal prevents the expulsion of air through the vent valve 223. The vent valve 223 may be opened again by moving the stopper 300 away from the cover 201 until the body 302 no longer contacts the cover 201.

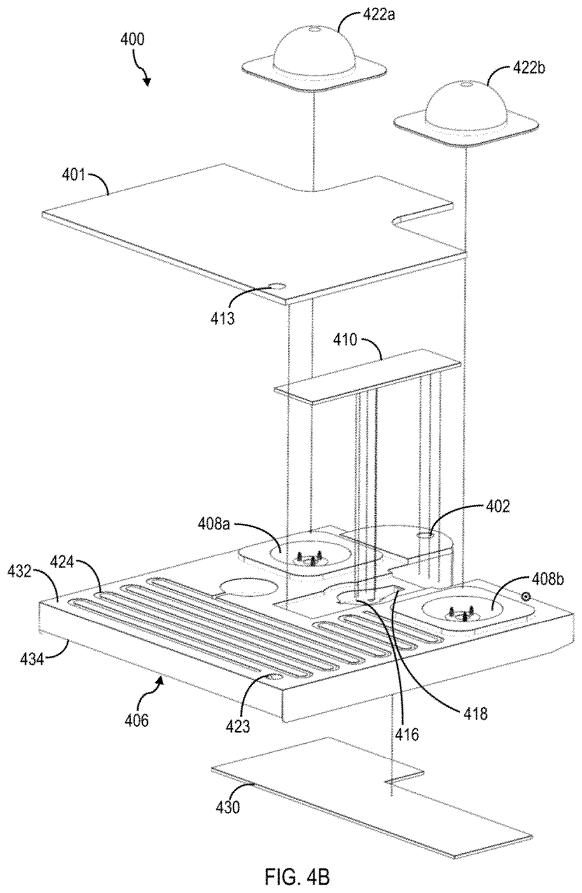

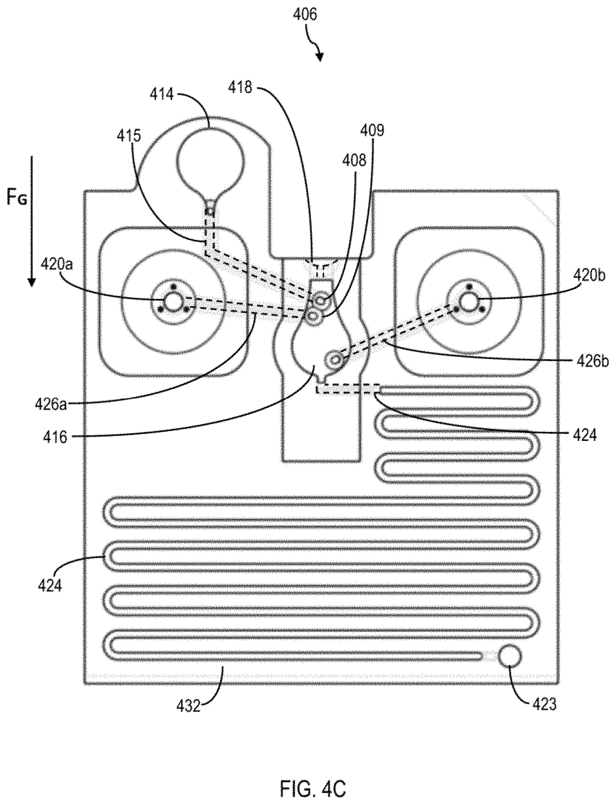

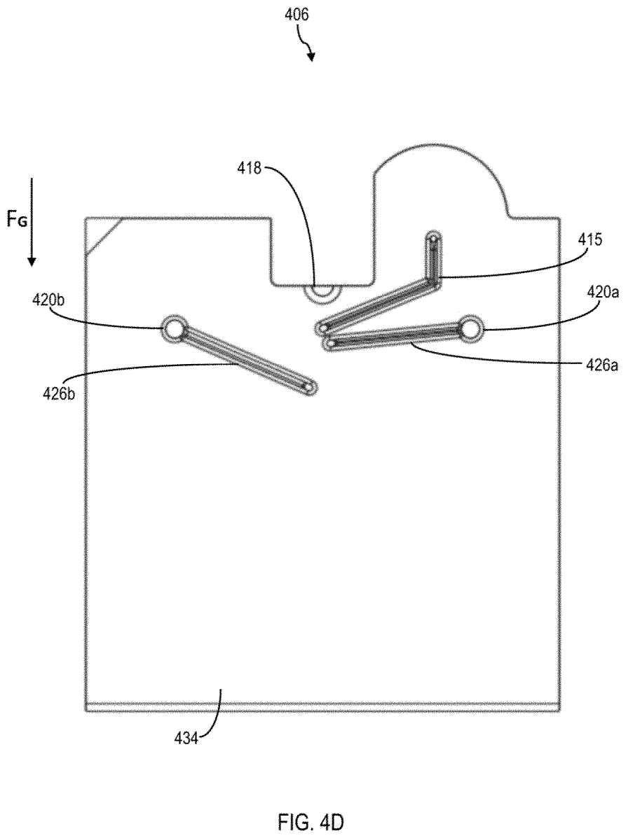

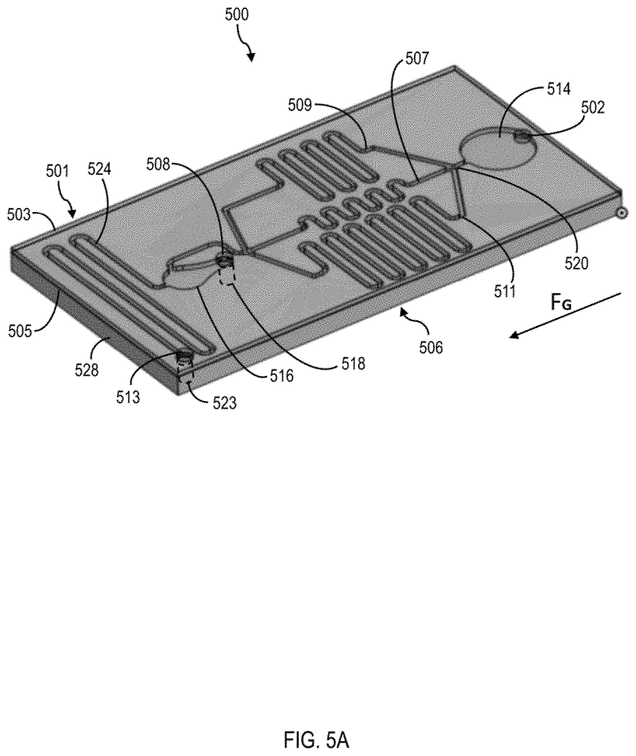

Referring to FIGS. 4A-4B, shown therein are perspective and exploded views, respectively, of a sample cartridge 400, according to an embodiment. The cartridge 400 may be the sample cartridge 106 in FIG. 1.

The sample cartridge 400 is substantially similar to the sample cartridge 200 in FIGS. 2A-2C. The cartridge 400 includes reagent blister packs 422a, 422b. The cartridge 400 includes a base 406 with a microfluidic system including an inlet 402, sensor reservoir 416, a top vent 418 a vent valve 423, wells 408a, 408b, and a waste channel 424. The movement of sample and reagents through the cartridge 422 is by force of gravity (indicated by arrow FG) and change in pressure in the same manner as described for cartridge 200. The opening and closing of the vent valve 423 may be achieved by the stopper 300 in the same manner as described for cartridge 200.

The cartridge 400 offers several manufacturing advantages to the cartridge 200. The cartridge includes an electrode 410 heat bonded directly to the base 406 to enclose the sensor reservoir 416, thus avoiding the need for an adhesive. Further, the channels 415, 426a, 426b, 424 in the base 406 are routed on both sides 432, 434 of the base 406 as shown in FIGS. 4C-4D (compared to the cartridge 200 wherein the channels are all routed on one side or embedded within the cartridge 200). This allows the base 406 to be easily manufactured as a single piece by injection molding.

Given there are channels on both sides 432, 434 of the base 406, a first hydrophilic cover 401 and a second hydrophilic cover 430 are used to enclose the channels on each side 434, 434 of the base 406, respectively. The first cover 401 and the second cover 430 are both hydrophilic to promote the flow of reagents and sample through the cartridge 400 along the channels and reservoirs formed between the base 406 and the first and second covers 401, 430.

A further benefit of the cartridge 400 is that fluid leakage from the sensor reservoir 416 back into the channels 415, 420a is prevented by positioning the channel openings 408, 409 toward the top of the sensor reservoir 416.

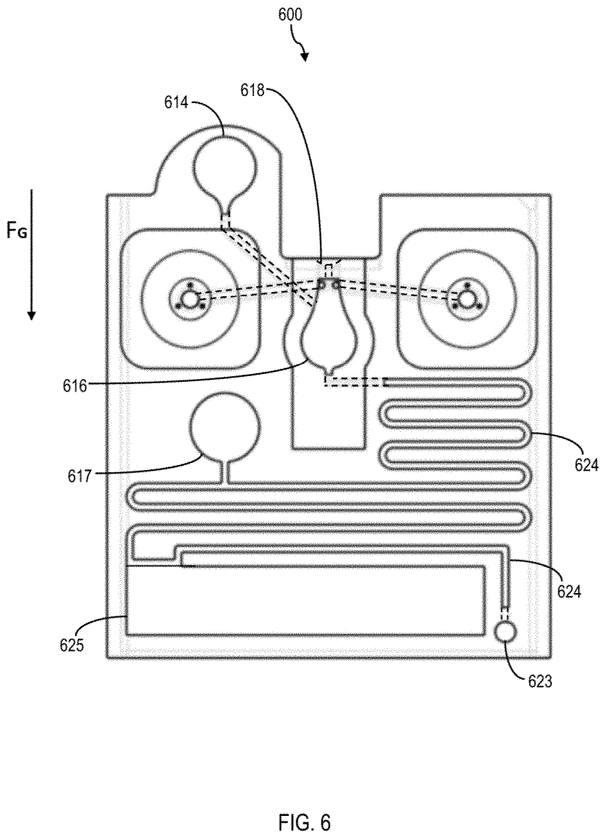

Now referring to FIG. 6, illustrated therein is a base 600 with a microfluidic system for use in a sample cartridge. The base 600 is substantially similar to the base 206 (FIGS. 2A-2C) and the base 406 (FIGS. 4A-4D). The movement of sample and reagents through the microfluidic system is by force of gravity (indicated by arrow FG) and change in pressure in the same manner as described for cartridge 200. The opening and closing of the vent valve 623 may be achieved by the stopper 300 in the same manner as described for the cartridge 200 (FIGS. 3A-3F).

The base 600 includes a microfluidic system including an inlet reservoir 614, sensor reservoir 616, a top vent 618 a vent valve 623, and a waste channel 624. The base 600 further includes an absorbent waste pad 625 in connection with the waste channel 624 upstream of the vent valve 623. Sample (and/or reagent as the case may be) flowing down the waste channel 624 is absorbed by the waste pad 625. The sample/reagent is retained by the waste pad 625, thus preventing the sample/reagent from overflowing the waste channel 624 and leaking out of the vent valve 623.

The base 600 further includes a diaphragm 617 in fluidic connection with the waste channel 624 upstream of the waste pad 625. The diaphragm 617 retains a volume of air within the microfluidic system (when a cover is attached to the base 600). The cover over the diaphragm 617 may be compressed (e.g. by actuator/servo 138 in FIG. 1) to push air from the diaphragm 617 to displace the fluid within the microfluidic system. Decompressing the cover over the diaphragm 617 pulls air from the microfluidic system into the diaphragm 617.

When the vent valve 623 is closed, compressing/decompressing the diaphragm 617 causes the fluid in the waste channel 624 and the sensor reservoir 616 to move bidirectionally as the fluid is displaced by air from the diaphragm 617. The diaphragm 617 may be successively compressed/decompressed to gently agitate the sample/reagent to improve mixing and diffusion without requiring a tenuous means of mechanical agitation (such as agitator 139 in FIG. 1) which may cause leakage of fluid from the microfluidic system.

While the cartridges 200, 400, 600 provide an improved cartridge format for point of care testing that abrogates the need for a pump or other means to move the sample through the cartridge during analyte measurement, it is desirable for a further simplified and cost-effective cartridge format for point of care testing. In particular, it is desirable to replace liquid phase reagents such as a wash buffer with lyophilized reagents. This offers several benefits.

Eliminating liquid reagents avoids the need for complex and relatively expensive to manufacture reagent blister packs on the cartridge. If blister packs are not needed for the cartridge, then the analyzer device may be simplified by eliminating the actuators/servos and electrical components for compressing the blister packs. Furthermore, lyophilized reagents may be reconstituted in the cartridge using the sample fluid itself so that no other fluid apart from the sample is needed to run a test. This can further simplify the manufacture of sample cartridges and make a point of care test easier to use.

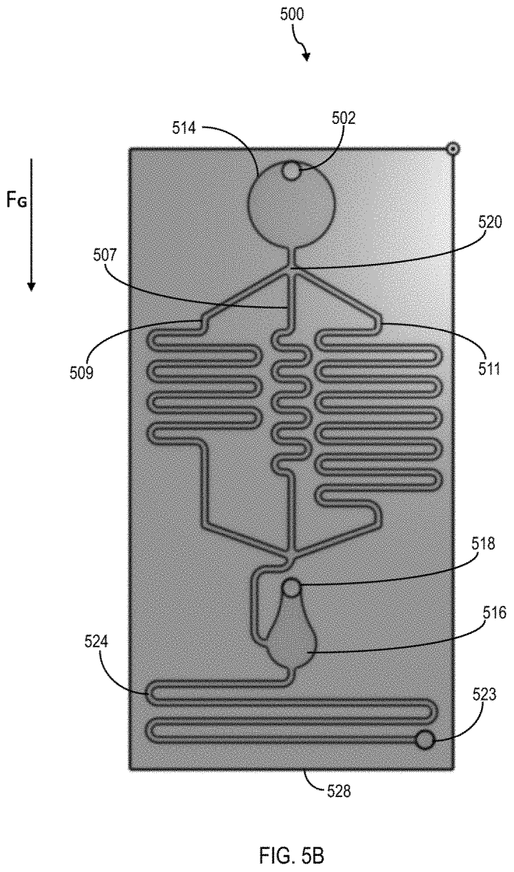

Referring to FIGS. 5A-5B, shown therein are perspective and top views, respectively, of a sample cartridge 500, according to an embodiment. The cartridge 500 may be the sample cartridge 106 in FIG. 1.

The cartridge 500 includes a base 506. The base 206 may be a single piece of PET or similar material formed by injection molding. The base 506 contains a microfluidic system of channels and reservoirs along which the sample and/or reagents move through the cartridge 500 during sample processing and analyte measurement. The microfluidic system is configured for a sample to pass through the cartridge 500 by force of gravity without requiring a pump, air line, or other means to transport the sample when the cartridge 200 is inserted into an analyzer device. The passage of the sample through cartridge 200 is controlled by release of air from the microfluidic system by the opening and closing of vent valves 518, 523 as described below.

The cartridge 500 includes an inlet 502 for receiving a sample of bodily fluid, for example, urine. Sample may be injected into cartridge 500 via the inlet 502 using a pipette, or the like. According to some embodiments, the inlet 502 may include a protuberance (not shown) which may be dipped into a volume of sample (e.g. urine in a cup). The protuberance may drain the sample into the inlet 502 by force of gravity when the cartridge 500 is inverted in the same manner as described for the cartridge 200, above. For reference, arrow FG points in the direction of gravitational force when the cartridge is inverted.

The cartridge 500 includes a hydrophilic cover 501 having a top surface 503 and a bottom surface 505. For ease of illustration, the cover 501 is depicted as being transparent; according to other embodiments the cover 501 may be opaque. The cover 501 includes a first recess 508 and a second recess 513 in the top surface 503 that extend to the bottom surface 505.

When the cover 501 is attached to the base 506, the bottom surface 505 encloses the channels and reservoirs within base 506. The bottom surface 505 is hydrophilic to promote the flow of reagents and sample through the cartridge 500 along the channels and reservoirs formed between the base 506 and the cover 501. The cover 501 may be bonded to the base 506 by adhesive. According to some embodiments, the cover 501 may be constructed of PET and heat bonded directly to the base 506 without adhesive.

Unlike the cartridges 200, 400 (FIGS. 2A-2C, 4A-4D) which have reagent blister packs for storing liquid reagents, the cartridge 500 includes lyophilized reagent within one or more channels 507, 509, 511. “Lyophilized” as used herein refers to freeze-dried, vacuum-dried, desiccated, powered or otherwise generally solid phase reagents.

The type and amount of lyophilized reagent in each channel 507, 509, 511 may be specific to the analyte measured or application the cartridge 500 is used for. For example, the lyophilized reagent may be a redox reagent including a redox probe; an enzyme-labelled secondary receptor and reactive reagent to be catalyzed by the enzyme (e.g. TMB and H₂O₂); an anticoagulant; a buffer, or other pH modulating reagent; or a dried label of the type described above.

According to other embodiments, the cartridge 500 may include fewer or more channels than the three channels 507, 509, 511 depicted, depending on the number of reagents required. Generally, one lyophilized reagent will be contained within one channel, thus the number of channels will equal the number of reagents.

The channels 507, 509, 511 may be of varying length. As shown, in order of increasing length: the channel 507<the channel 509<the channel 511. Accordingly, the same fluid, under force of gravity, will travel a longer distance, and take a longer time to travel along the channel 511 than the channel 509. Similarly, the fluid will take longer to travel along the channel 509 compared to the channel 507.

The base 506 includes an inlet reservoir 514 in fluidic connection with the inlet 502. Sample enters the cartridge 500 via the inlet 502 and collects in the inlet reservoir 514. Movement of the sample from the inlet reservoir 514 into the channels 507, 509, 511 and further downstream is regulated by a first vent valve 518 and a second vent valve 523 as described below.

The first vent valve 518 is aligned with the first recess 508 and first vent valve 518. The second vent valve 523 is aligned with the second recess 513 and second vent valve 523. The bottom surface 505 of the cover 501 seals the first and second vent valves 518, 523 when the cover 501 is attached to the base 506. The bottom surface 505 within the first and second recesses 508, 513 may be pierced (by, for example, a stopper 300 connected to an actuator in the analyzer device) to open the first and second vent valves 518, 523, respectively, in the same manner as described for cartridge 200 (see FIGS. 3A-3F).

The base 506 includes a sensor reservoir 516. The sensor reservoir 516 is in fluidic connection with the channels 507, 509, 511 and the first vent valve 518.

The cartridge 500 includes a sensor electrode (not shown for ease of illustration) exposed to the sample within the sensor reservoir 516. For example, may form a surface (or a cover at least a portion of the surface area) of the sensor reservoir 516 in the same manner as described for electrode 210 in cartridge 200 (see FIG. 2B). According to other embodiments, the electrode may be positioned entirely within the sensor reservoir 516. The electrode may be the electrode 110 in FIG. 1.

The base 506 includes a waste channel 524 for sample (and/or reagent as the case may be) to drain out of the sensor reservoir 516. The waste channel is 524 is in fluidic connection with the sensor reservoir 516 and the second vent valve 523.

Still referring to FIGS. 5A-5B, an exemplary implementation of the cartridge 500 for detecting presence of Luteinizing Hormone (LH) in a sample of urine is described. For brevity, only one assay format for electrochemical measurement of LH will be described. However, it is contemplated that the the cartridge 500 may be used in any of the assay formats and methods of analyte detection described for cartridge 200, above.

Urine is taken into the cartridge 500 via the inlet 502. The cartridge 500 is then inverted and connected (i.e. inserted) to an analyzer device (i.e. device 126 in FIG. 1). The cartridge 500 must be oriented with the inlet 502 upward and a bottom end 528 downward so that the sample travels through the cartridge 500 in a generally downward direction from the inlet 502, to the sensor reservoir 516, to the second vent valve 523 by force of gravity (indicated by arrow FG) and change of pressure within the microfluidic system.

Gravity drains the urine from the inlet 502 into the inlet reservoir 514. Urine collects in the inlet reservoir 514 and does not automatically proceed downward to the trifurcation point 520 by force of gravity since both the first and second vent valves 518, 523 are sealed by the cover 501 causing backpressure in the microfluidic system downstream of the inlet reservoir 514.

To commence downward flow of urine, the first vent valve 518 is opened. The first vent valve 518 may be opened by piercing the bottom surface 505 of the cover 501 within the first recess 508 using a first stopper/needle (i.e. stopper 300 in FIG. 3E) attached to an actuator in the analyzer device. When the first vent valve 523 is opened, the back pressure between the sensor reservoir 516 and the inlet reservoir 514 is relieved and the urine flows downward towards the trifurcation point 520 by force of gravity.

When the urine reaches the trifurcation point 520, the volume of urine will split and travel down each of the channels 507, 509, 511 by force of gravity. According to other embodiments, the trifurcation point 520 may include additional microfluidics (not shown) to split the sample evenly amongst the channels 507, 509, 511.

As the urine travels down the channels 507, 509, 511 lyophilized reagents within the channels 507, 509, 511, will be rehydrated/reconstituted by the urine. Given that the channel 507 is the shortest in length among the channels 507, 509, 511, urine flowing down the channel 507 will reach the sensor reservoir 516 first. Accordingly, the channel 507 may include a lyophilized label. In this case, the lyophilized label is a secondary antibody labelled with an enzyme that will react with a redox probe introduced later. Alternatively, the lyophilized label may be deposited on the surface of the sensor electrode within the sensor reservoir 516.

Once the urine reaches the sensor within the sensor reservoir 516, the first vent valve 518 is closed (see FIG. 3F). The closing of the first vent valve 518 creates backpressure upstream of the first vent valve 518 and stops the flow of urine along the channels 507, 509, 511.

The urine within the sensor reservoir 516 is left on the sensor surface for an incubation period (e.g. 30 mins). During this time two processes take place: (1) the urine reconstitutes the dried label (i.e. dried label in the sensor reservoir 516); and (2) the LH in the urine binds with the antibody on the electrode surface, and the reconstituted label, forming a sandwich complex at the electrode surface. The cartridge 500 may be agitated during the incubation period. Agitation may promote process (1) and/or (2).

Following the incubation period, urine is drained from the sensor reservoir 516 by opening the second vent valve 523. The second vent valve 523 may be opened by piercing the bottom surface 505 of the cover 501 within the second recess 513 using a second stopper/needle (see FIG. 3E) attached to an actuator in the analyzer device. When the second vent valve 523 is opened, the backpressure in the waste channel 524 is relieved and the urine drains from the sensor reservoir 516 into the waste channel 524 by force of gravity. Once the urine has drained into the waste channel 524, the second vent valve 523 is closed (see FIG. 3F), creating backpressure in the waste channel 524 and halting flow through the cartridge 500.

Next, the first vent valve 518 is reopened (see FIG. 3E) relieving the back pressure upstream of the first vent valve 518. This enables urine to flow downward along the channels 509, 511 and towards the reservoir by force of gravity (note: by this point, the channel 507 will be drained of urine).

Urine flowing down the channel 509 (the next shortest channel) will reach the sensor reservoir 516 next. The channel 509 includes lyophilized wash buffer that is reconstituted by the urine. The urine-wash buffer drains from the channel 509 into the sensor reservoir 516 and is prevented from flowing into the waste channel 524 by the backpressure in the waste channel 524 from the second vent valve 523 being closed. When the urine-wash buffer fills the sensor reservoir 516 to cover the electrode surface, the first vent valve 518 is closed, creating backpressure upstream of the first vent valve 518 and halting flow through the cartridge 500.

The urine-wash buffer is incubated on the electrode fora period of time (e.g. 10 seconds). The cartridge 500 may be agitated while the urine-wash buffer is incubated. The urine-wash buffer is then drained from the sensor reservoir 516 by opening the second vent valve 523 to relieve the pressure in the waste channel 524. Once the urine-wash buffer has completely drained from the sensor reservoir 516 into the waste channel 524 by force of gravity, the second vent valve 523 is closed, creating backpressure in the waste channel 524 and halting flow through the cartridge 500.

Next, the first vent valve 518 is reopened, relieving the back pressure upstream of the first vent valve 518. This enables urine to flow downward along the channel 511 towards the sensor reservoir 516 by force of gravity (note: by this point, the channels 507, 509 will be drained of urine).

The channel 511 includes lyophilized redox probe that is reconstituted by the urine. The urine-redox probe drains from the channel 511 into the sensor reservoir 516 and is prevented from flowing into the waste channel 524 by the backpressure in the waste channel 524 from the second vent valve 523 being closed. When the urine-redox probe fills the sensor reservoir 516 just enough to cover the electrode surface, the first vent valve 518 is closed, creating backpressure upstream of the first vent valve 518 and halting flow through the cartridge 500.

Following addition of the urine-redox probe, the measurement of LH by chronoamperometry (CA) is performed by applying a constant voltage across the electrode using the potentiostat (i.e. potentiostat 132 in FIG. 1) and measuring the current. The measurement(s) may be performed over a period of time. The cartridge 500 may be agitated during the measurement. After a measurement is obtained, the second vent valve 523 is opened to relieve the backpressure in the waste channel 524 and the urine-redox probe drains from the sensor reservoir 516 into the waste channel 524 by force of gravity.

While the above description provides examples of one or more apparatus, methods, or systems, it will be appreciated that other apparatus, methods, or systems may be within the scope of the claims as interpreted by one of skill in the art.

All patent applications cited herein are incorporated herein by reference in their entirety, except for any claims, definitions, subject matter disclaimers or disavowals, and except to the extent that the incorporated material is inconsistent with the express disclosure herein, in which case the language in this disclosure controls.

1. A cartridge for receiving a sample containing an analyte, the cartridge comprising:
an inlet for receiving the sample;
a reservoir in fluidic connection with the inlet, wherein the sample drains from the inlet into the reservoir by force of gravity;
a first channel having a first length, wherein the first channel is in fluidic connection with the inlet and the reservoir;
at least a second channel having a second length, wherein the second channel is in fluidic connection with the inlet and the reservoir; and
a first vent valve for regulating the pressure upstream of the reservoir,
wherein opening the first vent valve relieves the pressure in the first channel and the second channel thereby draining fluid from the inlet into the reservoir via the first channel and the second channel, by force of gravity.

2. The cartridge of claim 1, wherein sealing the first vent valve causes backpressure upstream of the reservoir thereby restricting the flow of fluid from the inlet into the reservoir by force of gravity.

3. The cartridge of claim 1, further comprising:
a waste channel in fluidic connection with the sensor reservoir; and
a second vent valve for regulating the pressure in the waste channel,
wherein opening the second vent valve relieves the pressure in the waste channel thereby draining fluid from the reservoir into the waste channel by force of gravity.

4. The cartridge of claim 3, wherein sealing the second vent valve causes backpressure in the waste channel thereby restricting the flow of fluid from the reservoir into the waste channel by force of gravity.

5. The cartridge of claim 1, further comprising:
at least a third channel having a third length wherein the third channel is in fluidic connection with the inlet and the reservoir,
wherein opening the first vent valve relieves the pressure in the third channel thereby draining fluid from the inlet into the reservoir via the third channel, by force of gravity.

6. The cartridge of claim 1, wherein the first channel contains a first lyophilized reagent.

7. The cartridge of claim 6, wherein the second channel contains a second lyophilized reagent.

8. The cartridge of claim 1, further comprising:
an electrochemical sensor for detecting the analyte, wherein the electrochemical sensor contacts the sample within the reservoir.

9. The cartridge of claim 8, further comprising:
electrical contacts for forming an interface with an analyzer device, wherein the electrical contacts are disposed on an exterior surface of the cartridge and relay signals between the electrochemical sensor and an analyzer device.

10. A system for point of care measurement of an analyte in a sample, the system comprising:
a cartridge, comprising:
an inlet for receiving the sample;
a reservoir in fluidic connection with the inlet, wherein the sample drains from the inlet into the reservoir by force of gravity;
a first channel having a first length, wherein the first channel is in fluidic connection with the inlet and the reservoir;
at least a second channel having a second length, wherein the second channel is in fluidic connection with the inlet and the reservoir;
cartridge contacts disposed on an exterior surface of the cartridge; and
a first vent valve for regulating the pressure upstream of the reservoir,
wherein opening the first vent valve relieves the pressure in the first channel and the second channel thereby draining fluid from the inlet into the reservoir via the first channel and the second channel, by force of gravity; and
an analyzer device, comprising:
device contacts for forming an interface with the cartridge contacts to relay signals between the cartridge and the analyzer device; and
a first stopper moveable between a first position for closing the vent and a second position for opening the first vent valve.

11. The system of claim 10, wherein the cartridge further comprises:
an electrochemical sensor for detecting the analyte, wherein the electrochemical sensor contacts the sample within the reservoir; and
the analyzer device further comprises:
a potentiostat for processing the signals from the electrochemical sensor to calculate a measurement of the analyte.

12. The system of claim 11, wherein the analyzer device further comprises:
a potentiostat for processing the signals from the electrochemical sensor to calculate a measurement of the analyte.

13. The system of claim 10, wherein the first stopper aligns with the first vent valve when the cartridge is connected to the analyzer device.

14. The system of claim 10, wherein the first stopper comprises:
a body, and a needle protruding from the body.

15. The system of claim 14, wherein in the first position, the body covers an opening of the first vent valve preventing the passage of air through the first vent valve.

16. The system of claim 14, wherein in the first position, the needle pierces a cover sealing the first vent valve.

17. The system of claim 14, wherein in the second position, the body uncovers the opening of the first vent valve allowing the passage of air through the first vent valve.

18. The system of claim 10, wherein the cartridge further comprises:
a waste channel in fluidic connection with the sensor reservoir; and
a second vent valve for regulating the pressure in the waste channel,
wherein opening the second vent valve relieves the pressure in the waste channel thereby draining fluid from the reservoir into the waste channel by force of gravity;
and wherein the analyzer device further comprises:
a second stopper moveable between a first position for closing the second vent valve and a second position for opening the second vent valve.

19. The system of claim 18, wherein the second stopper aligns with the second vent valve when the cartridge is connected to the analyzer device.

20. The system of claim 18, wherein the analyzer device further comprises:
control electronics configured to move the first stopper and the second stopper in a predefined sequence to process the sample.
